SEQUENCE LISTING (5 sequences)

SEQ ID NO: 1 - DNA, 67 bp, Artificial Sequence
  Synthetic DNA oligomer
cttcctcgaacaagggaggttcggaggagaacctctcgggggagaaagcgttggttaacgccggaca

SEQ ID NO: 2 - DNA, 63 bp, Artificial Sequence
  Syntheric DNA oligomer
tgtcttcgcctatagtagaaaccactagcacggacacatcgacctttctttcgactaagcacc

SEQ ID NO: 3 - DNA, 21 bp, Artificial Sequence
  Synthetic DNA oligomer
ggcgaagacaggtgcttagtc

SEQ ID NO: 4 - DNA, 15 bp, Artificial Sequence
  Peptide nucleic acid
tatagtagaaaccac

SEQ ID NO: 5 - DNA, 35 bp, Artificial Sequence
  Chimeric substrate
gatgtgtccgtgcnnnggttcgaggaagagatggc

(19) United States
(12) Patent Application Publication
(10) Pub. No.: US 2011/0294125 A1
(43) Pub. Date: Dec. 1, 2011

(54) COLORIMETRIC BIOSENSOR WITH ALLOSTERIC DNAZYME ACTIVATION AND ROLLING CIRCLE SIGNAL AMPLIFICATION

(75) Inventors: Yingfu Li, Dundas (CA); Monsur Ali, Hamilton (CA)

(73) Assignee: MCMASTER UNIVERSITY, Hamilton, ON (CA)

(21) Appl. No.: 13/141,007

(22) PCT Filed: Dec. 18, 2009
(86) PCT No.: PCT/CA2009/001847

§ 371 Date: Jun. 20, 2011

Related U.S. Application Data
(60) Provisional application No. 61/138,719, filed on Dec. 18, 2008.

(51) Int. Cl. C12Q 1/68; B82Y 15/00

(52) U.S. Cl. 435/6.11; 435/6.12; 977/810; 977/902

(57) ABSTRACT

The present disclosure includes a method of determining the presence of a target in a sample comprising an allosteric DNAzyme; rolling circle amplification dependent on the activity of the allosteric DNAzyme in the presence of target and a detection system. The methods further comprise quantifying the amount of target in the sample by comparing the detection with a control. Also included herein are kits for practicing the methods described herein and methods of designing biosensor systems.

### RELATED APPLICATIONS

This application claims the benefit of priority of copending U.S. provisional application No. 61/138,719 filed Dec. 18, 2008, the contents of which are incorporated herein by reference in their entirety.

### FIELD OF THE DISCLOSURE

The disclosure relates to a biosensor system and methods and kits including this system. In particular, the disclosure relates to an allosteric DNAzyme-rolling circle amplification-colorimetric biosensor system and methods and uses thereof.

### BACKGROUND OF THE DISCLOSURE

DNA aptamers and DNAzymes have recently received considerable attention in chemical biology research.^{[1]} These two classes of synthetic DNA molecules, which can be isolated from random DNA pools by in vitro selection,^{[2]} are regarded as attractive alternatives to antibodies and enzymes, particularly considering the fact that DNA has a greater chemical stability and can be easily prepared by automated synthesis. To date, a large number of DNA aptamers have been produced for recognition of targets from small molecules (such as ATP) to proteins (such as thrombin) to complex molecular assemblies like cells.^{[3]} Likewise, many DNAzymes have been made to catalyze diverse chemical reactions, such as cleavage and ligation of DNA and RNA.^{[4]} More recently, the concept of allosteric ribozyme^{[5]} has been adapted for the design of allosteric DNAzyme in which a DNA aptamer is joined to a DNAzyme such that the activity of the DNAzyme can only be activated by the ligand binding to the aptamer.^{[6]} Allosteric DNAzymes are interesting as biosensing tools because the molecular recognition event between an aptamer and its specific ligand can be translated into the activity of a DNAzyme for signal generation and amplification.

Rolling circle amplification (RCA) is a simple enzymatic process that can be used to generate, with the use of a short DNA primer and a circular template and under isothermal conditions, very long single-stranded DNA (ssDNA) molecules with tandem repeats.^{[7]} This reaction is carried out by special DNA polymerases, such as φ29 DNA polymerase, that have strand-displacement abilities. RCA has traditionally been used to achieve sensitive detection of DNA.^{[8]} In recent years, however, RCA has been expanded for detection of other targets, such as proteins and small molecules, through the use of DNA aptamers and allosteric DNAzymes.^{[9]} For example, the groups of Willner and Mao have recently applied the RCA technique to generate repetitive units of a reporter DNAzyme to achieve highly sensitive detection of DNA.^{[9e,f]} Ellington and coworkers have created a ligand-dependent ligase DNAzyme that can generate a circular DNA template to initiate an RCA process as a way to detect small molecule targets^{[9d]} and proteins.^{[9c]}

### SUMMARY OF THE DISCLOSURE

The present disclosure includes a method of determining the presence of a target in a sample comprising:

a) providing a substrate that comprises (i) a first DNA sequence that is complementary to a circular template, (ii) an RNA linkage and (iii) a second DNA sequence;

b) providing an allosteric DNAzyme that binds the substrate and masks the first DNA sequence in the absence of the target and that cleaves the substrate into a first and second DNA sequence in the presence of the target, releasing a primer comprising the first DNA sequence;

c) generating single stranded DNA molecules by rolling circle amplification in the presence of the circular template and the primer; and

d) detecting the single stranded DNA molecules generated in c);

wherein detection of single stranded DNA molecules in (d) indicates the presence of target in the sample.

In one embodiment, the detection of the single stranded DNA molecules in d) is compared to a control, wherein a difference or similarity in the detection between the sample and the control indicates the amount of target in the sample.

In another embodiment, the detection of the single stranded DNA molecules is by a colorimetric assay.

In one embodiment, detecting the single stranded DNA molecules in d) comprises:
d1) hybridizing the single stranded DNA molecules with a complementary peptide nucleic acid (PNA) to form DNA-PNA duplexes; and
d2) detecting the DNA-PNA duplexes with a duplex binding detection agent;

wherein detection of the DNA-PNA duplexes in (d2) indicates the presence of single stranded DNA molecules.

In another embodiment, detecting the single stranded DNA molecules in d) comprises
d1) hybridizing the single stranded DNA molecules with gold nanoparticles (AuNP) that are tethered to complementary DNA strands to form AuNP-DNA-DNA duplexes; and
d2) detecting the AuNP-DNA-DNA duplexes;

wherein detection of AuNP-DNA-DNA duplexes in (d2) indicates the presence of single stranded DNA molecules.

In yet another embodiment, detecting the single stranded DNA molecules in d) comprises
d1) binding the single stranded DNA molecules with gold nanoparticles (AuNP) to form AuNP-DNA complexes;
d2) detecting the AuNP-DNA complexes;

wherein detection of AuNP-DNA complexes in (d2) indicates the presence of single stranded DNA molecules.

Also included herein are kits for practicing the methods of the disclosure. In one embodiment, there is included a kit for determining the presence or quantity of a target, the kit comprising an allosteric DNAzyme that is activatable by the target; a substrate for the allosteric DNAzyme, wherein the substrate comprises a DNA primer that is releasable upon DNAzyme activity; a circular template that is amplifiable using the DNA primer; and a single stranded DNA detection system.

Further included herein is a method of designing a biosensor system for detecting a target comprising

a) preparing a substrate that comprises a first DNA molecule that is complementary to a circular template, an RNA linkage and a second DNA molecule; and

b) obtaining an allosteric DNAzyme that binds the substrate and masks the first DNA molecule in the absence of the target and that cleaves the substrate into the first and second DNA molecule in the presence of the target;

wherein the biosensor system comprises rolling circle amplification of the circular template using the cleaved first DNA molecule as a primer to generate single stranded DNA molecules and quantification of the single stranded DNA molecules by a colorimetric assay.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows a schematic of colorimetric detection of a specific target using (A) an RNA-cleaving allosteric DNAzyme, (B) RCA, and (C) PNA and DiSC2(5). When the target is present, the allosteric DNAzyme cleaves its substrate and generates a DNA primer to initiate RCA. The resultant long ssDNA forms duplex with a complementary PNA. DiSC2(5) binds PNA/DNA duplex and changes its color from blue to purple.

FIG. 2 shows (A) DNA sequences used for the test RCA reaction and (B) Color appearances and absorbance spectra of DiSC2(5) in the hybridization buffer alone (1), with the RCA product (2), and in mixture of the RCA product and the PNA probe (3).

FIG. 3 shows (A) Alternative conformations of ‘pH6-ET4’, an ATP-dependent allosteric DNAzyme. Insert: a PAGE gel showing the cleavage reaction in reaction buffer alone (lane 2), in the presence of GTP (lane 3) or ATP (lane 4). NR: no reaction. (B) Agarose gel of RCA products. RCA with authentic P1 (lane 1); DNAzyme/S1 incubated in the cleavage buffer alone (lane 2) as well as in the presence GTP (lane 3) and ATP (lane 4). Lane L: DNA ladders. (C) Color appearance when 1-4 was mixed with PNA/DiSC2(5). It should be noted that P1 carries a 2′,3′-cyclic phosphate which needs to be removed prior to RCA. This was achieved with the use of T4 polynucleotide kinase (PNK) which removes 2′,3′-cyclic phosphate.^{[16]}

FIG. 4 shows (A) 10% denaturing polyacrylamide gel electrophoresis of the radioactive RCA products when RCA-T was incubated with the cleavage mixture of pH6-ET4/S1 conducted in the presence of 0, 50, 100, 250, 500 and 1000 μl of ATP. (B) Color appearances of the samples in (A) in presence of 0.5% succ-β-cyD. (C) Plot of the color responses (CR) of the samples were obtained from the absorbance.

FIG. 5 shows cleavage of the substrate S1 by pH6-ET4 in presence of various ATP concentrations as indicated on top of the figure. 80 nM of S1 was treated with 2.5 μM of the enzyme pH6-ET4 in 50 μL of reaction volume for 10 min at room temperature. The reactions were quenched by adding 30 mM of EDTA and the DNA was isolated by ethanol precipitation. The reaction mixtures were dissolved in 45 μL of ddH₂O. 5 μL from each sample was transferred and applied for gel electrophoresis. The cleaved bands were visualized by Typhoon and quantified using ImageQuant software according to the previous method.^{[2]}

### DETAILED DESCRIPTION OF THE DISCLOSURE

A novel detection system which incorporates the technique of using an Allosteric DNAzyme in combination with rolling circle amplification (RCA), a peptide nucleic acid (PNA), and a colorimetric detection method that is triggered by events within the system has been developed.

This system provides a general strategy to devise biosensors, such as colorimetric biosensors, for the detection of a target analyte for which an allosteric DNAzyme, such as an RNA-cleaving allosteric DNAzyme, can be designed or created.

Accordingly, in one embodiment, there is included a method of determining the presence of a target in a sample comprising:

a) providing a substrate that comprises a DNA sequence that is complementary to a circular template; and

b) providing an allosteric DNAzyme that binds the substrate and masks the DNA sequence in the absence of the target and that releases the DNA sequence in the presence of the target;

c) generating single stranded DNA molecules by rolling circle amplification in the presence of the circular template, wherein the primer for the amplification is the DNA sequence; and

d) detecting the single stranded DNA molecules generated in c);

wherein detection of single stranded DNA molecules in (d) indicates the presence of the target in the sample.

In another embodiment, there is included a method of determining the presence of a target in a sample comprising:

a) providing a substrate that comprises (i) a first DNA sequence that is complementary to a circular template, (ii) an RNA linkage and (iii) a second DNA sequence; and

b) providing an allosteric DNAzyme that binds the substrate and masks the first DNA sequence in the absence of the target and that cleaves the substrate into the first and second DNA sequence in the presence of the target, releasing a primer comprising the first DNA sequence;

c) generating single stranded DNA molecules by rolling circle amplification in the presence of the circular template and the primer; and

d) detecting the single stranded DNA molecules generated in c);

wherein detection of single stranded DNA molecules in (d) indicates the presence of target in the sample.

In one embodiment, the detection of the single stranded DNA molecules in d) is compared to a control, wherein a difference or similarity in the detection between the sample and the control indicates the amount of target in the sample.

The term “control” as used herein refers to a positive or negative sample or a specific value or predetermined standard. For example, a positive control is a sample containing target or a sample or series of samples with known amounts of target and a negative control is a sample without target. The control can also be a predetermined set of values representing detection of particular amounts of target.

The term “mask” or “masking” as used herein refers to hiding or making unavailable the DNA primer. Thus, when the first DNA sequence is masked, the primer is unavailable and cannot initiate rolling circle amplification.

The term “nucleic acid” as used herein includes DNA and RNA and can be either double stranded or single stranded.

The term “DNAzyme” as used herein refers to a DNA molecule that has the ability to release the DNA molecule (primer) masked in the substrate, and includes, without limitation, DNAzymes with RNA-cleaving activity. The term “DNA aptamer” as used herein refers to short strands of nucleic acids that can adopt highly specific 3-dimensional conformations. Aptamers can exhibit high binding affinity and specificity to a target molecule. Both DNAzymes and DNA aptamers can be isolated from random DNA pools by in vitro selection methods known in the art.^{[2]}

The term “substrate” as used herein refers to the molecule that is being acted on by the DNAzyme cleaving activity. The substrate is designed so that upon DNAzyme activity, a short DNA molecule is generated that is complementary to a portion of a circular template such that it can act as a primer for rolling circle amplification. For an RNA-cleaving DNAzyme, the substrate comprises a RNA linkage that allows cleavage of the short DNA molecule.

The term “primer” as used herein refers to a nucleic acid sequence, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

The term allosteric DNAzyme as used herein refers to a molecule comprising both a DNAzyme and a DNA aptamer, wherein the DNAzyme is only active when the aptamer is bound by the target.

The target can include, but is not limited to, a small molecule, protein, bacteria fragment, or cell, or fragments thereof.

The term “rolling circle amplification” or “RCA” as used herein refers to the rolling amplification of circular DNA templates resulting in long single stranded DNA molecules. Conditions for rolling circle amplification are known in the art. In rolling circle amplification, the primer initiates amplification by a polymerase enzyme such as φ29 DNA polymerase that has strand displacement ability and which allows the production of long single strands of DNA to be produced.

A person skilled in the art would understand that there are numerous ways to detect the presence of single stranded DNA molecules in the sample after RCA and includes, without limitation, radioactive and colorimetric detection and/or quantification. For example, the generated DNA molecules can be labeled radioactively or the generated DNA molecules can be detected by hybridizing with a PNA or complementary DNA molecule and detecting duplexes formed. In one embodiment, the detection of the single stranded DNA molecules is quantitatively determined by ultraviolet or visible light spectroscopy. Quantitative analysis can be realized by recording the absorption spectra using a standard spectrophotometer. In another embodiment, the detection of the single stranded DNA molecules is qualitatively determined by a color change of the solution.

Accordingly, in one embodiment, the single-stranded DNA molecules resulting from the RCA, are hybridized to a complementary PNA sequence. In turn, this hybridization event is detected by the naked eye in the presence of a duplex-binding agent that changes color upon binding (DiSC2(5)) for example.

The term “peptide nucleic acid” or (PNA) as used herein refers to a DNA or RNA mimic whose backbone is composed of N-(2-aminoethyl)-glycine units linked by peptide bonds.

The term “hybridize” or “hybridizable” refers to the sequence specific non-covalent binding interaction with a complementary nucleic acid. In an embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The duplex-binding detection agent can be any molecule that changes colour upon binding to a DNA/PNA duplex. In one embodiment, 3,3′-diethylthiadicarbocyanine (DiSC2(5)) can be used, which is an organic dye that is known to change color from blue to purple upon binding to DNA/PNA duplex. In yet another embodiment, the method comprises measuring the absorbance of the DiSC2(5) dye in the presence of succinyl-β-cyclodextrin (Succ-β-CyD).

Accordingly, in another embodiment, detecting the amount of single stranded DNA molecules in d) comprises
d1) hybridizing the single stranded DNA molecules with a complementary peptide nucleic acid (PNA) to form DNA-PNA duplexes; and
d2) detecting the DNA-PNA duplexes with a duplex binding detection agent;

wherein detection of DNA-PNA duplexes in (d2) indicates the presence of single stranded DNA molecules.

In another embodiment, the single-stranded DNA molecules resulting from the RCA are hybridized with gold nanoparticles (AuNP) incorporated in the system, to provide a colorimetric sensor depending on the agglomeration of the associated DNA aptamer complexes. Stabilized gold nanoparticles and methods of making them are described in WO2008/119181, the contents of which are incorporated herein by reference. In an embodiment, the system includes aggregated AuNPs with tethered DNA strands that when bound to RCA products causes dispersion and a colour change from blue to red. In another embodiment, the system can use dispersed AuNPs which can aggregate in the presence of RCA products, causing color change from red to blue. In yet another embodiment, dispersed AuNPs bind RCA products via the formation of duplex structure, which can be cut by restriction enzymes and to cause AuNP aggregation and corresponding colour change under specific salt conditions.

Accordingly, in another embodiment, detecting the single stranded DNA molecules in d) comprises
(d1) hybridizing the single stranded DNA molecules with gold nanoparticles (AuNP) that are tethered to complementary DNA strands to form AuNP-DNA-DNA duplexes;
(d2) detecting the AuNP-DNA-DNA duplexes;

wherein detection of AuNP-DNA-DNA duplexes in (d2) indicates the presence of single stranded DNA molecules.

In yet another embodiment, detecting the amount of single stranded DNA molecules in d) comprises
(d1) binding the single stranded DNA molecules with gold nanoparticles (AuNP) to form AuNP-DNA complexes;
(d2) detecting the AuNP-DNA complexes;

wherein detection of AuNP-DNA complexes in (d2) indicates the presence of single stranded DNA molecules.

The amount of complexes detected or strength of signal, for example by colorimetric assay, indicates the amount of single stranded DNA molecules. The amount of single stranded DNA molecules correlates to the amount of target in the sample. This amount can be compared to a control or series of controls that represent known amounts of target. This allows the amount of target in the sample to be quantified based on the control value or values from which it is compared.

In another aspect, the present disclosure also includes a kit for determining the presence or quantity of a target, said kit comprising an allosteric DNAzyme that is activateable by the target; a substrate for the allosteric DNAzyme, wherein the substrate comprises a DNA primer that is releasable upon DNAzyme activity; a circular template that is amplifiable using the DNA primer; and a single stranded DNA detection system.

In one embodiment, the single stranded DNA detection system comprises a PNA complementary to the single stranded DNA generated from the circular template by the DNA primer and a duplex binding detection agent. In one embodiment, the duplex binding detection agent is DiSC2(5).

In another embodiment, the single stranded DNA detection system comprises AuNP particles that bind the single stranded DNA molecules. In yet another embodiment, the single stranded DNA detection system comprises AuNP particles tethered to DNA molecules that are complementary to the single stranded DNA generated from the circular template by the DNA primer.

In yet another embodiment, the kits disclosed herein also include, without limitation, instructions for use, reagents for DNAzyme activity, reagents for rolling circle amplification, such as dNTPs, DNA polymerase, including phi29 DNA polymerase and other agents commonly used in the processes described herein.

In yet another aspect, the present disclosure includes a method of designing a biosensor system for detecting a target comprising

a) preparing a substrate that comprises a first DNA molecule that is complementary to a circular template, an RNA linkage and a second DNA molecule; and

b) obtaining an allosteric DNAzyme that binds the substrate and masks the first DNA molecule in the absence of the target and that cleaves the substrate into the first and second DNA molecule in the presence of the target;

wherein the biosensor system comprises rolling circle amplification of the circular template using the cleaved first DNA molecule as a primer to generate single stranded DNA molecules and quantification of the single stranded DNA molecules.

In yet another aspect, the present disclosure includes a method of determining the presence or absence of a target comprising:

contacting a sample suspected of comprising the target with the detection system of the disclosure; and

determining the presence or absence of the target.

In one embodiment, determining the presence or absence of the target comprises observing or detecting a color change; wherein color change is indicative of the target.

In one embodiment, the sample is a biological, medical or environmental sample.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

### EXAMPLES

### A. Results

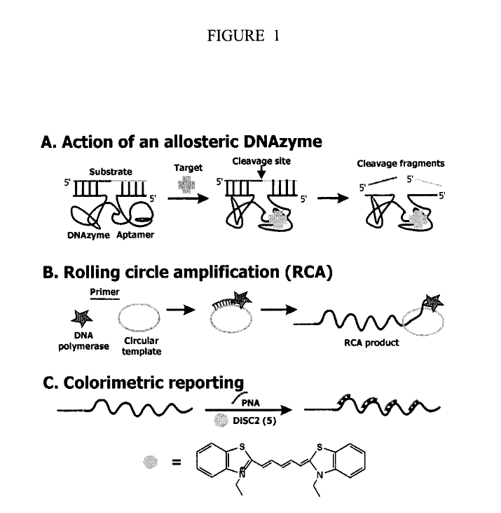

FIG. 1 illustrates an embodiment of the strategy of the present disclosure. Three designs are implemented: an RNA-cleaving allosteric DNAzyme, rolling circle amplification (RCA), and a colorimetric reporting mechanism based on a peptide nucleic acid (PNA) and an organic dye. In the presence of the intended target, the allosteric DNAzyme cleaves a special RNA-containing substrate and releases a DNA molecule that can be used by φ29 DNA polymerase as the primer to initiate RCA reaction for generating a long ssDNA. The RCA products will then be detected colorimetrically upon hybridization with a complementary PNA in the presence of DiSC2(5) (3,3′-diethylthiadicarbocyanine). PNA molecules are known to form highly stable duplex structures with complementary DNA sequences.^{[10]} It has also been reported that DiSC2(5) changes color from blue to purple upon binding to DNA/PNA duplex. This phenomenon has been used for colorimetric DNA detection.^{[11]} As is demonstrated below, by employing RCA, PNA and DiSC2(5), the binding event between an allosteric RNA-cleaving DNAzyme and its cognate target can be translated into a colorimetric signal visible to naked eyes.

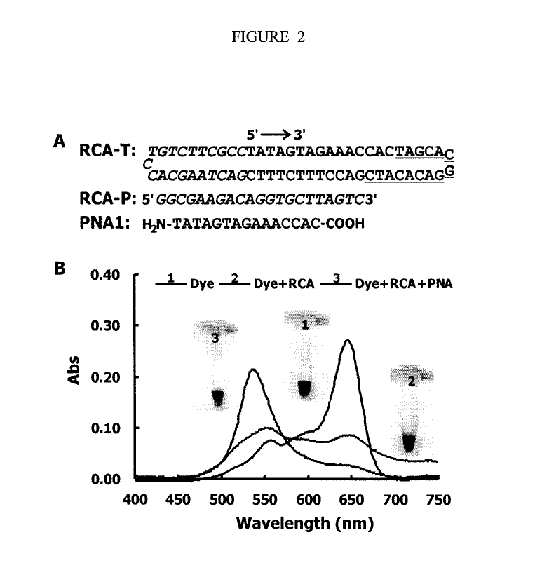

First, the colorimetric characteristics of DiSC2(5)-PNA probe were examined in the presence of RCA product. A circular ssDNA template, named ‘RCA-T’ (FIG. 2A) was synthesized using a previously reported protocol.^{[12]} RCA-T and the matching primer, ‘RCA-P’, were used to perform the RCA reaction as previously described.^{[13]} The details of this reaction and subsequent color development procedures are provided in the Electronic Supporting Information (ESI). The DiSC2(5) retained the blue color in the hybridization buffer (50 mM Tris-HCl, pH 7.5, and 100 mM NaCl) (FIG. 2B, tube 1). When DiSC2(5) was mixed with the RCA product, no significant color change was seen (tube 2). However, when treated with the RCA product in the presence of the complementary PNA (named ‘PNA1’; its sequence is given in FIG. 2A), the dye turned purple (tube 3). It should be noted that this dye slowly aggregated in the hybridization buffer with and without the RCA product; in contrast, no aggregation occurred in the RCA/PNA solution. It is also important to note that this dye can gradually turn into purple color in the PNA solution alone as in the PNA-DNA duplex. However, as previously reported,^{[11]} with heating and cooling, the purple color with the dye-PNA-DNA duplex appears faster (less than 1 min) than that of the dye-PNA alone (at least 5 min is required).

The absorbance spectra of the solutions 1-3 were also analyzed (FIG. 2B). The maximal absorbance of the dye in the hybridization buffer occurred at 646 nm. With the RCA product and PNA, the maximal absorbance was shifted to 537 nm. In comparison, the mixture of the dye and RCA product produced a broadened peak between 500-680 nm. These spectra were similar to those produced by others using PNA molecules hybridized to short DNA oligonucleotides.^{[11]} In short, the results above demonstrate that DiSC2(5)-PNA can indeed be used as colorimetric probe to visualize long ssDNA from RCA.

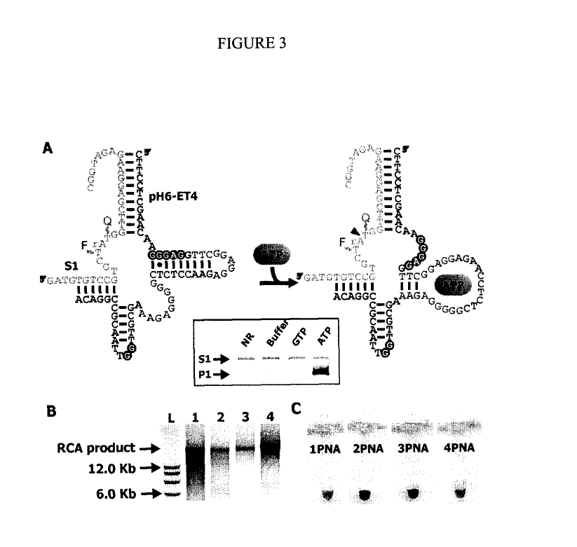

Next, RCA was carried out with ‘pH6-ET4’ (FIG. 3A), an ATP-sensing allosteric DNAzyme previously designed^{[6d]} from an RNA-cleaving and fluorescence-signaling DNAzyme named ‘pH6DZ1’^{[14]} and an ATP-binding DNA aptamer.^{[15]} pH6-ET4 can adopt two different conformations: in the absence of ATP, it adopts an inactive structure in which several catalytically important nucleotides form a short duplex with part of the aptamer sequence. In the presence of ATP, however, it switches to the active conformation because the aptamer domain folds into its binding structure and frees the catalytic core of the DNAzyme, which cleaves the RNA linkage (‘rA’, adenine ribonucleotide) embedded in an otherwise DNA substrate denoted ‘S1’. The cleavage event generates two separate DNA molecules, one of which (i.e. the 5′-cleaved fragment, denoted P1′) is designated to be the primer to initiate RCA. Key to this design is the placement of a masked primer that can only be retrieved for RCA upon the target-induced cleavage of S1 by the DNAzyme. Thus, the recognition of the target by the aptamer is translated into an RCA process.

It should be noted that pH6-ET4 could also report target binding through the generation of a fluorescence signal because of the fluorophore and quencher (F and Q in FIG. 3A) attached at the two T residues flanking the cleavage site. The activity of pH6-ET4 was confirmed to be dependent on the presence of ATP, as revealed by the PAGE (polyacrylamide gel electrophoresis) result shown as the insert of FIG. 3A: a much larger quantity of P1 (which is fluorescent and can be detected by fluorimaging) was produced in the mixture containing 1 mM ATP than in the mixture containing no ATP or 1 mM GTP (which has no affinity for the DNA aptamer).

P1 is designed to be complementary to part of the circular template RCA-T (underlined nucleotides in FIG. 2A). Four separate RCA reactions were performed and the RCA product was analyzed by agarose gel electrophoresis (FIG. 3B). When RCA-T was incubated with pre-cleaved P1 (a positive control), a significant amount of RCA product (bigger than the 12,000 base-pair ladder; lane L: DNA ladders) was observed (FIG. 3B, lane 1;). Expectedly, a similar amount of RCA product was seen when RCA-T was incubated with the reaction mixture of pH6-ET4/S1/ATP (lane 4). In contrast, only a small amount of RCA product was produced when ATP was omitted (lane 2) or replaced by (lane 3).

Each reaction mixture above was combined with PNA1 (5 μM) and the DiSC2(5) dye (50 μM). The resultant solutions were heated at 90° C. for 2 min and cooled to room temperature to facilitate hybridization between the PNA and the RCA product and color development (FIG. 3C). Both sample 2 (2PNA) and sample 3 (3PNA) retained the blue color of the dye; in contrast, sample 1 (1PNA) and sample 4 (4PNA) produced the expected purple color.

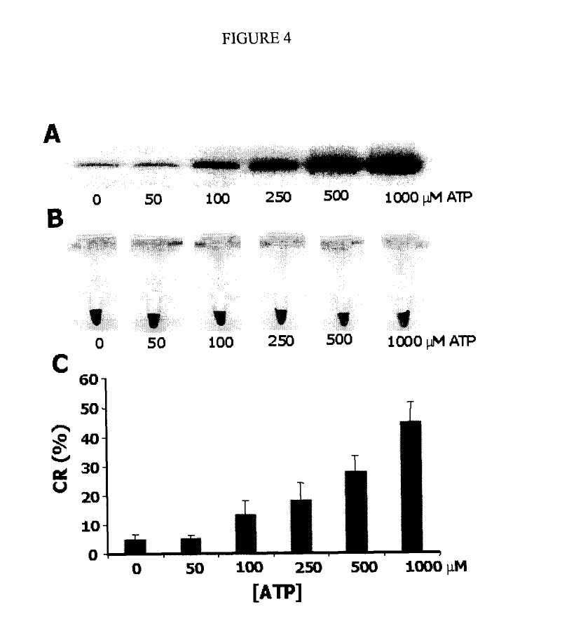

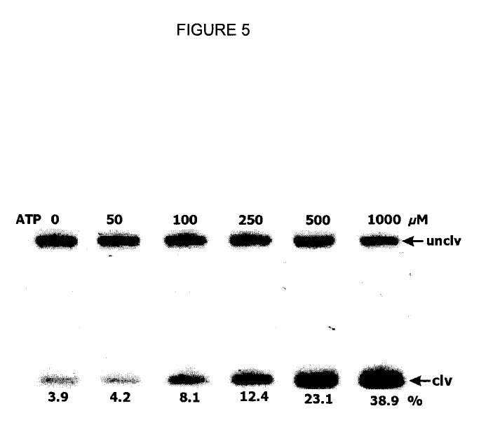

RCA reactions were performed using the cleavage products of pH6-ET4/S1 incubated at 0, 50, 100, 250, 500 and 1000 μM of ATP. These reactions were conducted in the presence of a trace amount of [α-³²P]-dGTP so that the amount of the RCA product measured by radioactivity could be correlated (FIG. 4A) with the purple color development (FIG. 4B). As expected, more RCA products and more intensive purple color were observed when more ATP was used. For comparison, a fluorescent gel image of the cleavage reaction at varied concentrations of ATP is also provided in ESI as FIG. 5, which showed that the cleavage product can be observed when 100 μM ATP was used, as observed with colorimetric results given in FIG. 4.

Finally, the color response (CR) of the above colorimetric samples was quantified by comparing absorbance (FIG. 4C) following a recently reported protocol^{[17]} (see ESI for details). As mentioned earlier, in a long incubation, DiSC2(5) with PNA alone can produce a purple color. To alleviate this problem, the absorbance of the dye in the RCA/PNA duplex was measured in the presence of succinyl-β-cyclodextrin (Succ-β-CyD).^{[18]} This reagent has been shown to interrupt the binding of the dye to the PNA alone without interfering with its ability to bind to the RCA/PNA duplex.^{[18]} Therefore the ratio of the absorbance of the dye at 535 nm vs. 647 nm can be directly related to the amount of RCA product produced at different amounts of ATP. Indeed, the CR profile of the RCA reaction mixtures at tested concentrations of ATP (FIG. 4C) reflects the increasing intensity of the purple color of relevant solutions (FIG. 4B).

In summary, demonstrated herein is a strategy of linking the action of an allosteric RNA-cleaving DNAzyme to RCA for the production of long ssDNA molecules so that colorimetric sensing can be achieved through hybridization of a complementary PNA sequence in the presence of a duplex-binding dye such as DiSC2(5). This approach can work as a general strategy to devise colorimetric biosensors for the detection of a target analyte for which an allosteric RNA-cleaving DNAzyme can be designed or created.

### B. Materials and Methods

MATERIALS. DNA oligonucleotides were synthesized using automated DNA synthesis (Integrated DNA Technologies, Coralville, Iowa) following the standard phosphoramidite chemistry, and purified by 10% denaturing PAGE before use. The fluorescently labeled DNA oligonucleotides were obtained from Invitrogen (Carlsbad, Calif.) and purified by HPLC. Peptide nucleic acid, PNAP, was purchased from Bio-Synthesis Inc (Lewisville, Tex.). T4 DNA ligase, Phi29 DNA polymerase and T4 polynucleotide kinase (PNK) were purchased from MBI Fermentas (Burlington, Canada). [α-³²P]dGTP was purchased from Perkin Elmer (Woodbridge, ON, Canada). Agarose was obtained from Bioshop (Burlington, Canada). Water used in this work is double-distilled and autoclaved. The autoradiogram and fluorescent images of gels were obtained using Typhoon 9200 variable mode imager (GE healthcare) and analyzed using ImageQuant software (Molecular Dynamics). Unless otherwise noted, all other materials were purchased from Sigma (Oakville, Canada) and used without further purification.

ROLLING CIRCLE AMPLIFICATION PROCEDURE. The typical RCA reaction was conducted in a volume of 50 μL. 10 pmol of RCA-T (circularized DNA template) was mixed with 10 pmol of RCA-P (RCA primer) in 40 μL of H₂O. This solution was heated to 90° C. for 1 min and cooled to room temperature for 10 min. To this mixture 5 μL of 10×RCA buffer (330 mM Tris acetate, pH 7.9 at 37° C., 100 mM magnesium acetate, 660 mM potassium acetate, 1% (v/v) Tween 20, 10 mM DTT, provided by MBI Fermentas) was added, followed by the addition of dNTP mix so that the final concentration of each of the dNTP was 500 μM. Finally, 1 μL of Phi29 DNA polymerase (10 U) was added, the volume was adjusted to 50 μL with H₂O. The reaction mixture was incubated at 30° C. for 3 h before heating at 65° C. for 10 min to stop the reaction. The RCA product was isolated by ethanol precipitation and dissolved in 50 μL of the hybridization buffer containing 50 mM Tris-HCl, pH 7.5, 100 mM NaCl.

COLORIMETRIC DETECTION OF THE RCA PRODUCT. Three 0.5-mL microcentrifuge tubes were marked as 1, 2, and 3. In tube 1, 20 μL of the hybridization buffer was added; in other two tubes, 10 μL of the above RCA product was taken and diluted to 20 μL with the hybridization buffer. 1.5 μL (100 pmol) of PNA1 was added to tube 3. Then, all the tubes were heated to 90° C. for 1 min and cooled to room temperature for 10 min. Thereafter, 1 μL of DiSC2(5) (1 mM stock, dissolved in methanol) was added to each tube and the mixture was heated for 2 min and allowed to cool to room temperature. The color images were captured during the cooling process (at ˜1-2 min) by digital camera (Panasonic, LUMIX) or by scanning using HP ScanJet 3570C.

CLEAVAGE REACTION OF pH6-ET4/S1. The cleavage reaction was conducted with a protocol adapted from our previous report (reference 6d in the main text). 200 pmol of pH6-ET4 was added to 10 pmol of the substrate (S1) (in 24.5 μL H₂O), followed by the addition of 0.5 μL of ATP (100 mM). The cleavage reaction was initiated by adding 25 μL of the 2× reaction buffer (100 mM MES, pH 6.0, 100 mM NaCl, 16 mM MgCl₂, 4 mM NiCl₂) at room temperature. After 10 min, the reaction was quenched by adding 3 μL of EDTA (0.5 M, pH 8.0). The DNA was isolated by ethanol precipitation. Two control experiments were also conducted in parallel. In the first control, 0.5 μL of H₂O was added to replace ATP, and in the second control, 0.5 μL of GTP (100 mM) was added. 5 μL from each reaction mixture was analyzed by 10% denaturing PAGE to confirm the cleavage. A fluorescence image of the PAGE gel is given as the insert in FIG. 3A.

REMOVAL OF THE 2′,3′-CYCLIC PHOSPHATE. The cyclic phosphate of the 5′-cleaved fragment was removed following our previously reported protocol using PNK.^{[1,2]} Briefly, the pellet of the above cleavage reaction mixture was diluted to 20 μL with ddH₂O, followed by addition of 2 μL of the 10×PNK buffer and 1 μL of PNK. The reaction mixture was incubated at 37° C. for 60 min. The reaction mixture was heated at 90° C. for 5 min to inactivate the enzyme. After cooling to room temperature, the DNA product was isolated by ethanol precipitation.

ROLLING CIRCLE AMPLIFICATION OF THE CLEAVAGE PRODUCT AND ENSUED COLOR DEVELOPMENT. The precipitated DNA above was dissolved in 40.0 μL of H₂O, to which 10 pmol of RCA-T was added. The sample was heated to 90° C. for 1 min and cooled to room temperature for 10 min, followed by the addition of 5 μL of 10×RCA buffer, dNTP mixture, Phi29 DNA polymerase, as described above. The reaction volume was adjusted to 50 μL with H₂O. The remaining steps were identical to the ones described above.

### Cleavage Reaction at Different Concentrations of ATP, and Relevant RCA, Color Development, UV and CR Analysis

CLEAVAGE, RCA AND GEL ELECTROPHORESIS. The cleavage reactions and removal of the cyclic phosphate groups were performed in the same way as described above except that the cleavage reactions were conducted at 0, 50, 100, 250, 500 and 1000 μM concentration. The RCA reactions were conducted in the same way as above in 50 μL reaction volume with the inclusion of a trace amount of α-³²P-[dGTP]. After ethanol precipitation, the RCA product was dissolved in 50 μL hybridization buffer, 10 μL of which was applied to the 10% denaturing PAGE.

COLOR DEVELOPMENT, UV MEASUREMENT AND CR ANALYSIS. The RCA product (10 μL) from each sample was transferred to a microcentrifuge tube and the volume was adjusted to 25 μL with the hybridization buffer. 1 μL of PNAP (150 pmol) and 1 μL of DiSC2(5) was added. In order to stabilize the color for UV analysis, 1 μL of 15% Succ-β-CyD (in 10% methanol) was added to each sample, which was then heated and cooled to room temperature. The color was captured with Panasonic LUMIX. For UV measurement, each sample was diluted to 500 μL with the hybridization buffer containing 0.5% Succ-β-CyD before heating and cooling. The absorbance was taken with Cary300 UV/Vis spectrophotometer.

CR CALCULATION. The color response, CR, of each sample was calculated from the UV absorbance as follows:

CR=[(B₀−B₁)/B₀]×100%

Where B=A_{blue}/(A_{blue}+Aₚᵤᵣₚₗₑ); A is the absorbance at either the “blue” component in the UV-Vis spectrum (ca. 647 nm) or “purple” component (ca. 535 nm); B0 is the purple/blue ratio of the control sample (dye alone); and B1 is the value obtained from the samples at different amount of ATP (0, 50, 100, 250, 500 and 1000 μM).

### REFERENCES

[1] a) N. K. Navani, Y. Li, Curr. Opin. Chem. Biol. 2006, 10, 272-281; b) J. F. Lee, G. M. Stovall, A. D. Ellington, Curr. Opin. Chem. Biol. 2006, 10, 282-289.
[2] a) A. D. Ellington and J. W. Szostak, Nature 1990, 346, 818-822; b) C. Tuerk and L. Gold, Science 1990, 249, 505-510.
[3] a) M. Famulok, G. Mayer, M. Blind, Acc. Chem. Res. 2000, 33, 591-599; b) D. S. Wilson, J. W. Szostak, Annu. Rev. Biochem. 1999, 68, 611-647; c) D. Shangguan, Y. Li, Z. Tang, Z. C. Cao, H. W. Chen, P. Mallikaratchy, K. Sefah, C. J. Yang, W. Tan, Proc. Natl. Acad Sci. U.S. A. 2006, 103, 11838-11843.
[4] a) J. C. Achenbach, W. Chiuman, R. P. Cruz, Y. Li, Curr. Pharm. Biotechnol. 2004, 5, 321-336; b) S. K. Silverman, Nucleic Acids Res 2005, 33, 6151-6163.
[5] a) G. A. Soukup, R. R. Breaker, Proc. Natl. Acad. Sci. U.S.A. 1999, 96, 3584-3589; b) R. R. Breaker, Curr. Opin. Biotechnol. 2002, 13, 31-39.
[6] a) M. Levy, A. D. Ellington, Chem Biol. 2002, 9, 417-426; b) S. H. Mei, Z. Liu, J. D. Brennan, Y. Li, J. Am. Chem. Soc. 2003, 125, 412-420; c) J. C. Achenbach, R. Nutiu, Y. Li, Anal. Chim. Acta 2004, 534, 41-51; d) Y. Shen, W. Chiuman, J. D. Brennan, Y. Li, Chem Bio Chem. 2006, 7, 1343-1348; e) W. Chiuman, Y. Li, PLoS ONE 2007, 2, e1224.
[7] a) A. Fire, S. Xu, Proc. Natl. Acad. Sci. U.S.A. 1995, 92, 4641-4645; b) D. Liu, S. L. Daubendiek, M. A. Zillman, K. Ryan, E. T. Kool, J. Am. Chem. Soc. 1996, 118, 1587-1594.
[8] a) P. M. Lizardi, X. Huang, Z. Zhu, P. Bray-Ward, D. C. Thomas, D. C. Ward, Nat Genet. 1998, 19, 225-232; b) C. Larsson, J. Koch, A. Nygren, G. Janssen, A. K. Raap, U. Landegren, M. Nilsson, Nat. Methods 2004, 1, 227-232.
[9] a) D. A. Di Giusto, W. A. Wlassoff, J. J. Gooding, B. A. Messerle, G. C. King. Nucleic Acids Res. 2005, 33, e64; b) L. Yang, C. W. Fung, E. J. Cho, A. D. Ellington. Anal. Chem. 2007, 79, 3320-3329; c) L. Zhou, L.-J. Ou, X. Chu, G.-L. Shen, R.-Q. Yu, Anal. Chem. 2007, 79, 7492-7500; d) E. J. Cho, L. Yang, M. Levy, A. D. Ellington. J. Am. Chem. Soc. 2005, 127, 2022-2023; e) Z. Cheglakov, Y. Weizmann, B. Basnar, I. Willner. Org. Biomol. Chem. 2007, 5, 223-225; f) Y. Tian, Y. He, C. Mao. Chem Bio Chem 2006, 7, 1862-1864. f) Zhao W, Ali M M, Brook M A, Li Y, Angew Chem Int Ed. 2008, 47, 6330-6337.
[10] a) M. Egholm, O. Buchardt, L. Christensen, C. Behrens, S. M. Freier, D. A. Driver, R. H. Berg, S. K. Kim, B. Norden, P. E. Nielsen, Nature 1993, 365, 566-568. 6) O. Buchardt, M. Egholm, R. H. Berg, P. E. Nielsen, Trends Biotechnol. 1993, 11, 384-6.
[11] a) S. A. Kushon, J. P. Jordan, J. L. Seifert, H. Nielsen, P. E. Nielsen, B. A. Armitage, J. Am. Chem. Soc. 2001, 123, 10805-10813; b) M. Komiyama, S. Ye, X. Liang, Y. Yamamoto, T. Tomita, J-M. Zhou, H. Aburatani, J. Am. Chem. Soc., 2003, 125, 3758-3762; c) I. Dilek, M. Madrid, R. Singh, C. P. Urrea, B. A. Armitage. J. Am. Chem. Soc. 2005, 127, 3339-3345. d) L. M. Wilhelmsson, B. Norden, K. Mukherjee, M. T. Dulay, R. N. Zare, Nucleic Acids Res. 2002, 30, e3.
[12] L. P. Billen, Y. Li, Bioorg. Chem. 2004, 32, 582-598.
[13] a) W. Zhao, Y. Gao, S. A. Kandadai, M. A. Brook, Y. Li. Angew. Chem. Int. Ed. 2006, 45, 2409-2413; b) W. Zhao, Y. Gao, M. A. Brook, Y. Li, Chem. Commun. 2006, 3582-3584; c) M. M. Ali, S. Su, C. D. Filipe, R. Pelton, Y. Li, Chem Commun. 2007, 4459-61.
[14] a) Z. Liu, S. H. Mei, J. D. Brennan, Y. Li, J. Am. Chem. Soc. 2003, 125, 75397545; b) Y. Shen, J. D. Brennan, Y. Li, Biochemistry 2005, 44, 12066-12076.
[15] a) D. E. Huizenga, J. W. Szostak, Biochemistry 1995, 34, 656-665; b) R. Nutiu, Y. Li, Angew. Chem. Int. Ed. 2005, 44, 1061-1065.
[16] S. A. Kandadai, W. Chiuman, Y. Li, Chem. Commun. 2006, 2359-2361
[17] A. Reichert, J. O. Nagy, W. Spevak, D. Charych J. Am. Chem. Soc. 1995, 117, 829-830.
[18] T. Tedeschi, S. Sforza, S. Ye, R. Corradini, A. Dossena, M. Komiyama, R. Marchelli, J. Biochem. Biophys. Methods, 2007, 70, 735-741.

1. A method of determining the presence of a target in a sample comprising:
a) providing a substrate that comprises (i) a first DNA sequence that is complementary to a circular template, (ii) an RNA linkage and (iii) a second DNA sequence; and
b) providing an allosteric DNAzyme that binds the substrate and masks the first DNA sequence in the absence of the target and that cleaves the substrate into a first and second DNA sequence in the presence of the target, releasing a primer comprising the first DNA sequence;
c) generating single stranded DNA molecules by rolling circle amplification in the presence of the circular template and the primer; and
d) detecting the single stranded DNA molecules generated in c);
wherein detection of single stranded DNA molecules in (d) indicates the presence of target in the sample.

2. The method of claim 1, wherein the detection of the single stranded DNA molecules in d) is compared to a control, wherein a difference or similarity in the detection between the sample and the control indicates the amount of target in the sample.

3. The method of claim 1, wherein the detection of the single stranded DNA molecules is by a colorimetric assay.

4. The method of claim 1, wherein detecting the single stranded DNA molecules in d) comprises:
d1) hybridizing the single stranded DNA molecules with a complementary peptide nucleic acid (PNA) to form DNA-PNA duplexes; and
d2) detecting the DNA-PNA duplexes with a duplex binding detection agent;
wherein detection of the DNA-PNA duplexes in (d2) indicates the presence of single stranded DNA molecules.

5. The method of claim 4, wherein the duplex binding detection agent comprises 3,3′-diethylthiadicarbocyanine (DiSC2(5)).

6. The method of claim 5, wherein the presence of DNA-PNA duplexes is indicated by a change in colour.

7. The method of claim 5, wherein detection or measurement of the absorbance of the DiSC2(5) dye is in the presence of succinyl-β-cyclodextrin (Succ-β-CyD).

8. The method of claim 1, wherein detecting the single stranded DNA molecules in d) comprises
d1) hybridizing the single stranded DNA molecules with gold nanoparticles (AuNP) that are tethered to complementary DNA strands to form AuNP-DNA-DNA duplexes; and
d2) detecting the AuNP-DNA-DNA duplexes;
wherein detection of AuNP-DNA-DNA duplexes in (d2) indicates the presence of single stranded DNA molecules.

9. The method of claim 8, wherein the presence of AuNP-DNA-DNA duplexes is indicated by a change in colour.

10. The method of claim 1, wherein detecting the single stranded DNA molecules in d) comprises
d1) binding the single stranded DNA molecules with gold nanoparticles (AuNP) to form AuNP-DNA complexes;
d2) detecting the AuNP-DNA complexes;
wherein detection of AuNP-DNA complexes in (d2) indicates the presence of single stranded DNA molecules.

11. The method of claim 10, wherein the presence of AuNP-DNA duplexes is indicated by a change in colour.

12. The method of claim 1, wherein the target is a small molecule, a protein, a bacterial fragment or a cell, or fragment thereof.

13. A kit for determining the presence or quantity of a target, said kit comprising an allosteric DNAzyme that is activatable by the target; a substrate for the allosteric DNAzyme, wherein the substrate comprises a DNA primer that is releasable upon DNAzyme activity; a circular template that is amplifiable using the DNA primer; and a single stranded DNA detection system.

14. The kit of claim 13, wherein the single stranded DNA detection system comprises a peptide nucleic acid (PNA) complementary to the single stranded DNA generated from the circular template by the DNA primer and a duplex binding detection agent.

15. The kit of claim 14, wherein the duplex binding detection agent is 3,3′-diethylthiadicarbocyanine (DiSC2(5)).

16. The kit of claim 15, further comprising succinyl-β-cyclodextrin (Succ-β-CyD).

17. The kit of claim 13, wherein the single stranded DNA detection system comprises AuNP particles that bind the single stranded DNA molecules.

18. The kit of claim 13, wherein the single stranded DNA detection system comprises AuNP particles tethered to DNA molecules that are complementary to the single stranded DNA generated from the circular template by the DNA primer.

19. A method of designing a biosensor system for detecting a target comprising
a) preparing a substrate that comprises a first DNA molecule that is complementary to a circular template, an RNA linkage and a second DNA molecule; and
b) obtaining an allosteric DNAzyme that binds the substrate and masks the first DNA molecule in the absence of the target and that cleaves the substrate into the first and second DNA molecule in the presence of the target;
wherein the biosensor system comprises rolling circle amplification of the circular template using the cleaved first DNA molecule as a primer to generate single stranded DNA molecules and quantification of the single stranded DNA molecules by a colorimetric assay.
